(19) United States
(12) Patent Application Publication
(10) Pub. No.: US 2013/0245376 A1
(43) Pub. Date: Sep. 19, 2013

(54) TUBE ASSEMBLY FOR ENDOSCOPE AND ATTACHING METHOD

(71) Applicant: FUJIFILM CORPORATION, Tokyo (JP)

(72) Inventor: Masatoshi OKU, Ashigarakami-gun (JP)

(73) Assignee: FUJIFILM CORPORATION, Tokyo (JP)

(21) Appl. No.: 13/799,128

(22) Filed: Mar. 13, 2013

(30) Foreign Application Priority Data
Mar. 14, 2012 (JP) 2012-056777

(51) Int. Cl. A61B 1/00; A61B 1/005

(52) U.S. Cl. CPC A61B 1/00071; A61B 1/005; A61B 1/0011
USPC 600/129; 29/432; 228/165

(57) ABSTRACT

A tube assembly for an endoscope includes a steering device and a flexible tube device on a proximal side. The tube assembly includes an inner sleeve. An outer sleeve receives one end portion of the inner sleeve in an axial direction, for connection of the flexible tube device to the steering device. A flow opening is formed in a portion of the outer sleeve disposed around the inner sleeve. A distribution groove is formed between an outer wall surface of the inner sleeve and an inner wall surface of the outer sleeve, to extend from the flow opening. A seal cavity is formed in the inner and outer sleeves to extend from the distribution groove in a direction away from the flow opening. Flowing solder material is supplied in the flow opening, charged in the distribution groove and the seal cavity, for attaching the outer sleeve to the inner sleeve.

### BACKGROUND OF THE INVENTION

1. Field of the Invention

The present invention relates to a tube assembly for an endoscope and an attaching method. More particularly, the present invention relates to a tube assembly for an endoscope in which plural sections are connected to one another by use of inner and outer sleeves, and strength in attachment between the inner and outer sleeves with sealing material can be stable, and an attaching method for the tube assembly.

2. Description Related to the Prior Art

An endoscope is a medical instrument used widely for diagnosis and treatment. The endoscope includes an elongated tube assembly for entry in a body cavity, and a handle manually operated by a doctor or operator. The elongated tube assembly includes a tip device, a steering device and a flexible tube device. The tip device includes an imaging unit with an image sensor such as a CCD, CMOS sensor. The steering device includes a plurality of link elements connected in series.

Various internal elements are incorporated in the elongated tube assembly, including wire devices, a signal cable, a light guide device, an instrument channel, a fluid supply channel and the like. The wire devices are operable for pull in response to steering wheels on the handle. The signal cable is connected with the imaging unit. The instrument channel is used for entry of a treatment device. The fluid supply channel supplies air and water. Ends of the internal elements are fixedly mounted on the tip device. In the course of coupling the steering device to the flexible tube device, the internal elements are previously contained in those. If screws are used for connecting the steering device to the flexible tube device, elements such as anti-drop rings or seals for preventing drop of the screws are entered together, structurally to enlarge a diameter of the elongated tube assembly. The anti-drop rings may project radially to a considerable extent, and interfere with a wall of the body cavity in the course of the entry of the endoscope.

To solve the problem with the screws, JP-A 7-084192 discloses a connection structure without screws. A connector for connecting the tip device to the steering device is suggested, is disposed between a protection mesh or net of the steering device and an outer sleeve of the tip device, and includes an annular groove. The protection mesh covers an outer surface of the steering device, and has an end portion entered in the annular groove. The connector is fitted in the outer sleeve. Flow openings are formed in the outer sleeve. Flowing solder material as sealing material is injected through the flow openings for filling the annular groove to connect the tip device to the steering device firmly.

In JP-A 7-084192, the presence of the protection mesh between the connector and the outer sleeve reduces a distribution channel for the solder. It is impossible to check whether the annular groove is filled with the solder. A problem arises in that the strength of the soldering is uneven according to instability in a range of flow of the solder. The decrease in the strength due to the shortage of the sealing material occurs not only with the solder but also with an adhesive agent of a liquid state. Furthermore, fluidity of the flowing solder material should be maintained for the purpose of penetrating the solder through the protection mesh. A soldering iron must contact portions of the soldering. Heat generated by the soldering may damage the internal elements.

### SUMMARY OF THE INVENTION

In view of the foregoing problems, an object of the present invention is to provide a tube assembly for an endoscope in which plural sections are connected to one another by use of inner and outer sleeves, and strength in attachment between the inner and outer sleeves with sealing material can be stable, and an attaching method for the tube assembly.

In order to achieve the above and other objects and advantages of this invention, a tube assembly for an endoscope is provided, including a first section, and a second section disposed to extend from the first section to a proximal side in an axial direction. The tube assembly includes an inner sleeve. An outer sleeve receives one end portion of the inner sleeve in the axial direction, for connection of the second section to the first section. At least one flow opening is formed through the outer sleeve, and opposed to an outer wall surface of the inner sleeve upon receiving the inner sleeve in the outer sleeve. A distribution groove is formed in at least one of the outer wall surface of the inner sleeve and an inner wall surface of the outer sleeve, to extend from the flow opening according to a circumferential direction. At least one seal cavity is formed in at least one of the outer wall surface of the inner sleeve and the inner wall surface of the outer sleeve, to extend from the distribution groove to a sleeve end of the outer sleeve in an externally open form. Sealing material is supplied in the flow opening, charged in the distribution groove and the seal cavity, for attaching the outer sleeve to the inner sleeve.

The distribution groove is annular.

The distribution channel includes a first distribution groove formed in the outer wall surface of the inner sleeve. A second distribution groove is formed in the inner wall surface of the outer sleeve, and disposed in alignment with the first distribution groove upon mounting the inner sleeve in the outer sleeve.

The seal cavity includes a first seal cavity formed in the inner sleeve to extend from the first distribution groove in the axial direction. A second seal cavity is formed in the outer sleeve to extend along the first seal cavity from the second distribution groove to a sleeve end of the outer sleeve.

The first seal cavity includes an open end area disposed outside the sleeve end of the outer sleeve.

The distribution channel includes plural distribution channels arranged adjacently to one another in the axial direction.

In another preferred embodiment, the distribution channel is in a helical shape with plural turns.

In one preferred embodiment, the at least one flow opening is a plurality of flow openings formed in the outer sleeve and arranged substantially equidistantly in a circumferential direction thereof.

The at least one seal cavity is plural seal cavities of which a number is equal to or larger than a number of the flow opening.

The at least one flow opening is plural flow openings, and the seal cavities are offset from the flow openings in a circumferential direction of the outer sleeve.

In still another preferred embodiment, the seal cavities are aligned with the flow opening in the axial direction.

The first section is a steering device, and the second section is a flexible tube device.

The inner sleeve is disposed at an end of the steering device, and the outer sleeve is disposed at an end of the flexible tube device.

The steering device includes first to Nth link elements, arranged serially in the axial direction toward the flexible tube device, and connected with one another movably. The inner sleeve is the Nth link element.

Furthermore, a cover sleeve is mounted on at least the steering device, for covering the inner and outer sleeves.

Also, an attaching method for an endoscope having a tube assembly is provided, the tube assembly including a tip device having an internal element, a steering device, mounted on a proximal side of the tip device, for steering operation, and a flexible tube device disposed to extend from the steering device to a proximal side in an axial direction, wherein an inner sleeve is used to constitute a proximal end of the steering device, an outer sleeve is used to constitute a distal end of the flexible tube device, one end portion of the inner sleeve is receivable in the outer sleeve in the axial direction, for connection of the flexible tube device to the steering device. The attaching method includes a step of forming at least one flow opening through the outer sleeve, the flow opening being opposed to an outer wall surface of the inner sleeve upon receiving the inner sleeve in the outer sleeve. A distribution groove is formed in at least one of the outer wall surface of the inner sleeve and an inner wall surface of the outer sleeve, to extend from the flow opening according to a circumferential direction. At least one seal cavity is formed to extend from the distribution groove to a sleeve end of the outer sleeve in an externally open form upon receiving the inner sleeve in the outer sleeve. A cable and an elongated element are penetrated through the steering device and the flexible tube device in connection with the internal element. The inner sleeve is mounted in the outer sleeve upon penetrating the cable and the elongated element through the flexible tube device. After the mounting step, sealing material is supplied in the flow opening, to charge the distribution groove and the seal cavity with the sealing material.

The sealing material is flowing solder material.

Consequently, it is possible to keep strength in attachment between the inner and outer sleeves with sealing material in a stable state, because the sealing material can spread in the distribution channel and the seal cavity for tight contact.

### BRIEF DESCRIPTION OF THE DRAWINGS

The above objects and advantages of the present invention will become more apparent from the following detailed description when read in connection with the accompanying drawings, in which:

FIG. 1 is a plan illustrating an endoscope;

FIG. 2 is a vertical section illustrating an elongated tube assembly;

FIG. 3 is a perspective view illustrating a solder joint;

FIG. 4 is a perspective view illustrating the solder joint in an assembled state;

FIG. 5 is a perspective view illustrating a state of the solder joint filled with solder;

FIG. 6 is a vertical section, partially broken, illustrating the same as FIG. 5;

FIG. 7 is a perspective view illustrating another preferred solder joint having three distribution channels in an inner sleeve;

FIG. 8 is a vertical section, partially broken, illustrating the solder joint;

FIG. 9 is a perspective view illustrating one preferred solder joint having three helical distribution channels in an inner sleeve;

FIG. 10 is a vertical section, partially broken, illustrating the solder joint;

FIG. 11 is a perspective view illustrating still another preferred solder joint having four pairs of seal cavities;

FIG. 12 is a cross section illustrating the solder joint;

FIG. 13 is a perspective view illustrating a state of the solder joint filled with the solder;

FIG. 14 is a perspective view illustrating one preferred solder joint having two flow openings;

FIG. 15 is a cross section illustrating the solder joint;

FIG. 16 is a perspective view illustrating a state of the solder joint filled with the solder.

### DETAILED DESCRIPTION OF THE PREFERRED EMBODIMENT(S) OF THE PRESENT INVENTION

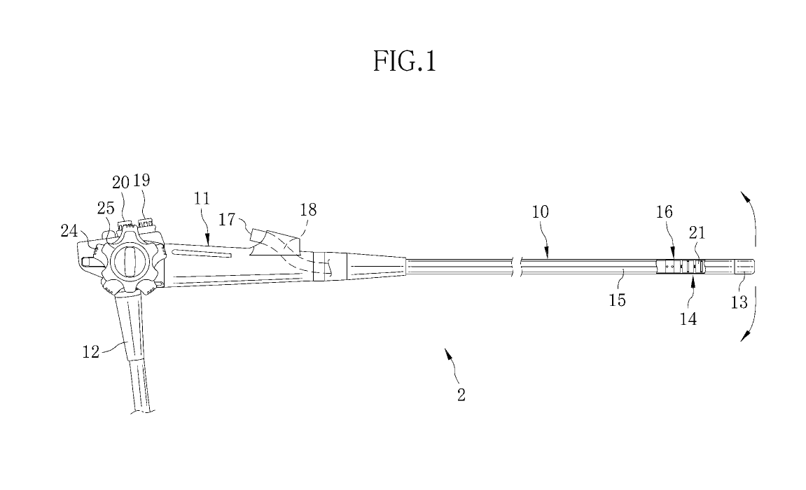

In FIG. 1, an endoscope 2 includes an elongated tube assembly 10 or insertion tube, a handle 11 and a universal cable 12. The elongated tube assembly 10 is entered in a body cavity of a patient. The handle 11 is mounted on a proximal end of the elongated tube assembly 10. The universal cable 12 extends from the handle 11. A processing apparatus (not shown) is connected by the universal cable 12 with the endoscope 2. The elongated tube assembly 10 includes a tip device 13, a steering device 14 and a flexible tube device 15. The steering device 14 is mounted on a proximal end of the tip device 13, and bendable for steering. The flexible tube device 15 extends from a proximal end of the steering device 14. The flexible tube device 15 is as long as several meters for reach of the tip device 13 to a target site in the body cavity. A solder joint 16 or connector connects the steering device 14 to the flexible tube device 15.

An imaging unit is incorporated in the tip device 13, and includes a lens system and an image sensor such as a CCD, CMOS sensor. Object light from an object in the body cavity is received through the lens system, and is detected by the imaging unit for imaging, to generate an image signal. A signal cable (not shown) is penetrated through the elongated tube assembly 10 and the handle 11, and transmits the image signal from the image sensor a processing apparatus through the universal cable 12. The processing apparatus processes the image signal in image processing of various functions. A monitor display panel (not shown) is driven by the processing apparatus to display the image.

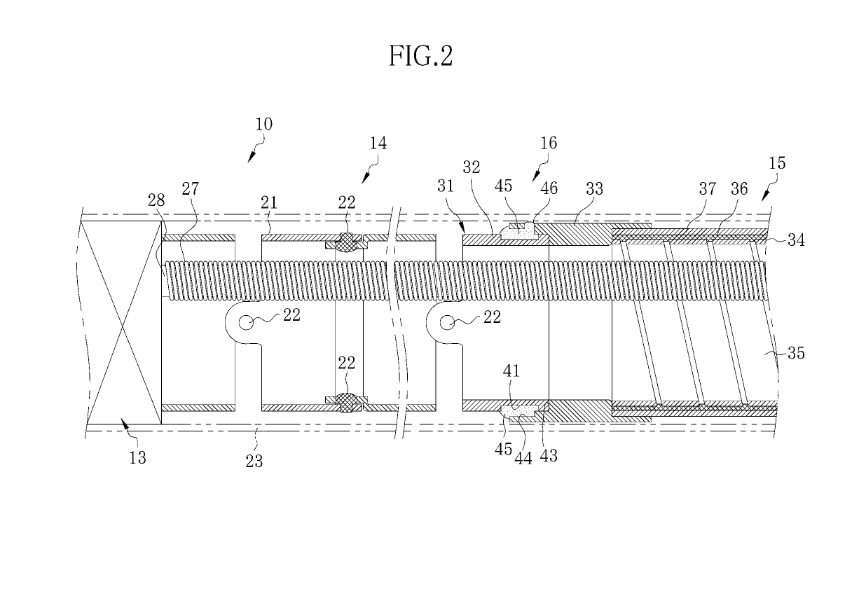

At least one lighting window is formed in the tip device 13. There is a light source apparatus (not shown) in connection with the universal cable 12. In FIG. 2, the endoscope 2 has a wire coil 27 or winding, and a light guide device 28 extending through the wire coil 27. The light guide device 28 guides light from the light source apparatus through the elongated tube assembly 10 and the handle 11, and emits the light toward an object in the body cavity.

An instrument opening 17 is formed in the handle 11, and used for penetration of a medical instrument for treatment, for example, forceps, injection needle and the like. An instrument channel 18 is formed through the elongated tube assembly 10, and communicates with the instrument opening 17. A distal instrument opening (not shown) is formed in the tip device 13. The instrument channel 18 extends to the distal instrument opening.

A fluid supply button 19 and a suction button 20 are disposed on the handle 11. A fluid channel (not shown) is formed through the elongated tube assembly 10. The fluid supply button 19 is depressible for supply of air or water through the fluid channel. A nozzle (not shown) is formed in the tip device 13. The air or water from the fluid channel is ejected by the nozzle. The suction button 20 is depressed for suction and discharge of body fluid or other waste fluid from the body cavity through the instrument channel 18.

In FIG. 2, the steering device 14 includes a plurality of link elements 21 and pins 22. The link elements 21, for example 16 links, are connected serially by the pins 22 in a pivotally movable manner. A flexible cover sleeve 23 of rubber covers the link elements 21 for protection. A first one of the link elements 21 at a distal end is attached firmly to the tip device 13. A proximal link element 31 is included in the link elements 21 in the steering device 14, and constitutes an inner sleeve 32 of the solder joint 16. A steering wheel 24 is disposed on the handle 11, and is rotatable for bending the steering device 14 up and down. A steering wheel 25 on the handle 11 is rotatable for bending the steering device 14 to the right and left. The tip device 13 can be oriented in a desired direction in the body cavity.

The flexible tube device 15 includes a winding 35 of a strip 34 of metal, a mesh sleeve 36 and encapsulant 37. The mesh sleeve 36 is constituted by mesh of wires of metal. The encapsulant 37 is disposed around the mesh sleeve 36 for encapsulation in a form of a jacket or sheath. The solder joint 16 has an outer sleeve 33, to which the encapsulant 37 at an end of the flexible tube device 15 is connected in a fluid tight manner.

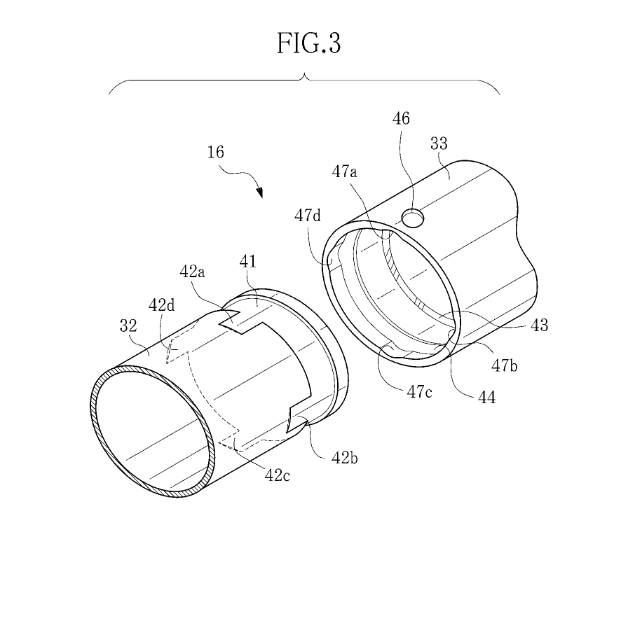

The solder joint 16 is described now by referring to FIGS. 3-6. A first distribution groove 41 or distribution channel is formed in the outer wall surface of the inner sleeve 32 and extends circumferentially in FIG. 3. A first seal cavity 42a or sub groove is formed in the inner sleeve 32, and extends from the first distribution groove 41 in a direction (proximal) away from a sleeve end of the inner sleeve 32. Also, first seal cavities 42b, 42c and 42d or sub grooves are formed in the inner sleeve 32 and arranged with the first seal cavity 42a equidistantly in relation to a circumferential direction.

A receiving surface 43 is formed inside the outer sleeve 33 between sections of large and small inner diameters. The sleeve end of the inner sleeve 32 becomes engaged with the receiving surface 43 when the inner sleeve 32 is mounted in the outer sleeve 33. Also, a second distribution groove 44 or distribution channel is formed in an inner wall surface of the outer sleeve 33, and extends circumferentially.

A flow opening 46 or gate is formed in the outer sleeve 33. Flowing solder material 45 such as molten solder or solder paste (sealing material) is injected into the flow opening 46. The flow opening 46 communicates with the second distribution groove 44. A second seal cavity 47a or sub groove is formed in the outer sleeve 33, and extends from the second distribution groove 44 to a sleeve end of the outer sleeve 33. Also, second seal cavities 47b, 47c and 47d or sub grooves are formed in the outer sleeve 33, and are arranged with the second seal cavity 47a equidistantly in relation to a circumferential direction.

An imaging unit is incorporated in the tip device 13. Various elongated elements are penetrated through the steering device 14 and the flexible tube device 15, including a signal cable from the imaging unit, the light guide device 28, the instrument channel 18, a fluid supply channel, a control wire and the like.

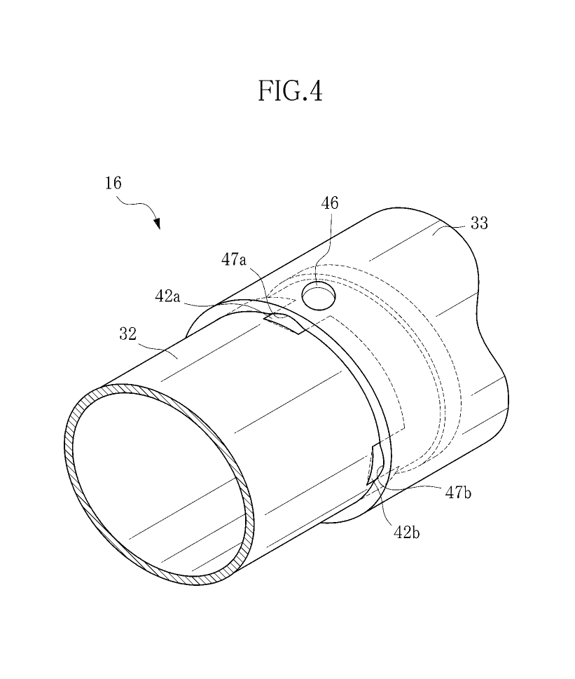

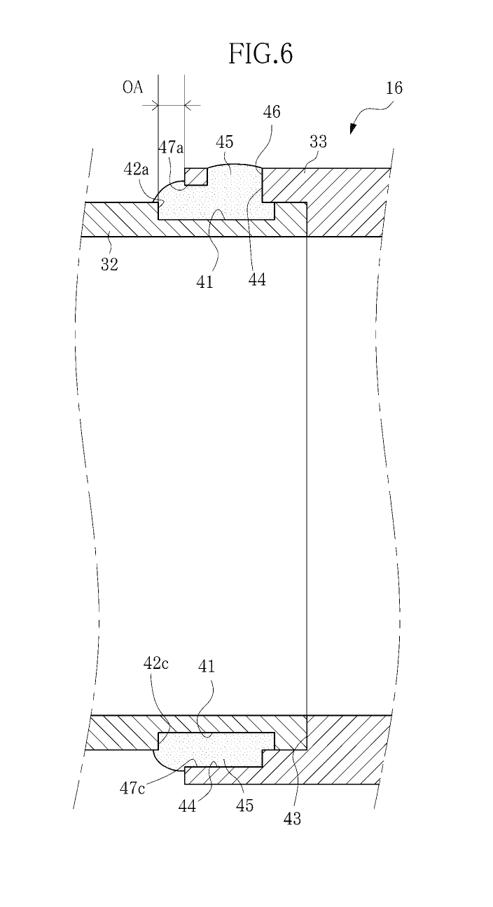

In FIG. 4, the inner sleeve 32 at the end of the steering device 14 and the outer sleeve 33 at the end of the flexible tube device 15 are combined together. The first distribution groove 41 is radially aligned with the second distribution groove 44 to define a distribution channel (one space). The first seal cavities 42a-42d are open to the outside with open end areas having a size OA in FIG. 6. A space between the inner and outer sleeves 32 and 33 is locally larger at the first and second distribution grooves 41 and 44, and utilized for injection of the solder 45 in the circumferential direction.

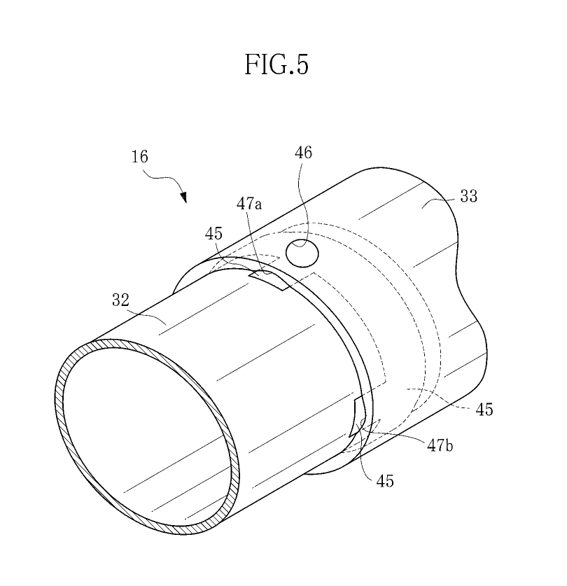

The solder 45 is injected through the flow opening 46 into the first and second distribution grooves 41 and 44, and then flows into the first seal cavity 42a and the second seal cavity 47a. The solder 45 flows further in the circumferential direction in the first and second distribution grooves 41 and 44, and comes in the first seal cavities 42b and 42d and the second seal cavities 47b and 47d as illustrated in FIG. 5. When the first and second distribution grooves 41 and 44 are filled with the solder 45 completely, the solder 45 enters the first seal cavity 42c and the second seal cavity 47c as illustrated in FIG. 6.

Thus, it is possible to check a condition of flow of the solder 45 into the first seal cavities 42a-42d and the second seal cavities 47a-47d. Strength of soldering can be maintained. Also, fluidity of the solder 45 can be high in addition to higher stability in an amount of the solder 45, because an area of flow of the solder 45 within the solder joint 16 is made large. It is unnecessary to apply a soldering iron (or hot bar apparatus) to the flow opening 46 for the purpose of increasing fluidity of the solder 45. An increase in the temperature of the solder joint 16 due to the soldering iron can be prevented, so that damages to internal elements can be prevented. Even if the solder 45 moves away from the surfaces of the first and second distribution grooves 41 and 44, the solder 45 in the solid state keeps the inner sleeve 32 on the outer sleeve 33 without drop, because the solder 45 keeps the first and second distribution grooves 41 and 44 positioned without offsetting.

Note that each of the first seal cavities 42a-42d has an open end area disposed outside the outer sleeve 33 with the size OA. However, the first seal cavities 42a-42d can be covered by the outer sleeve 33 without an open end area.

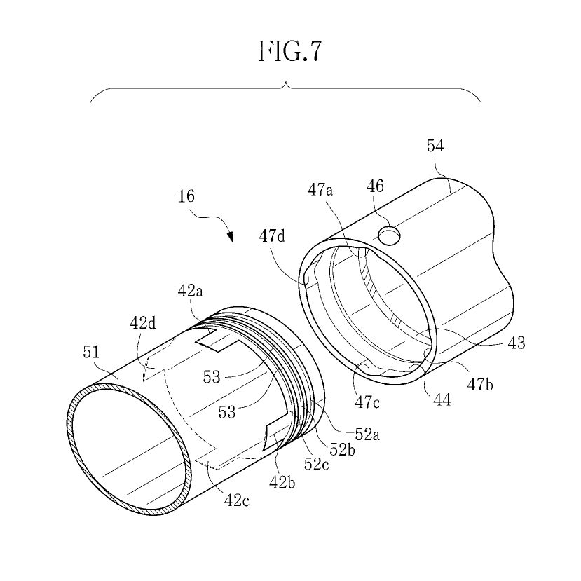

In FIG. 7, a second preferred tube assembly of the invention is illustrated. The first embodiment is repeated but with a difference in that an inner sleeve 51 has three first distribution grooves 52a, 52b and 52c or distribution channels in contrast with the first distribution groove 41 of the inner sleeve 32. Elements similar to those of the first embodiment are designated with identical reference numerals.

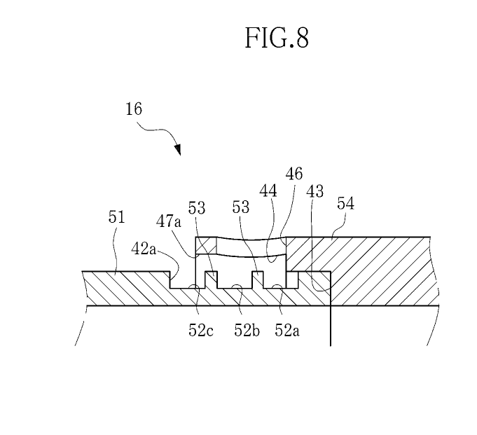

In FIG. 8, annular projections 53 or wall ridges are defined between the first distribution grooves 52a-52c. An outer sleeve 54 has the flow opening 46. A height of the annular projections 53 is predetermined so that the flow opening 46 communicates with the first seal cavity 42a. In the same manner as the first embodiment, the inner sleeve 51 is mounted in the outer sleeve 54. The solder 45 is injected into the flow opening 46, flows through the first distribution grooves 52a-52c and reaches the first seal cavities 42a-42d.

Strength of soldering can be maintained, because an area of the contact with the solder 45 can be large with the shape of the first distribution grooves 52a-52c. Also, the annular projections 53 of the first distribution grooves 52a-52c can function for reinforcement. A problem of a small strength may occur in the first embodiment in that walls are formed with a limited thickness under the first and second distribution grooves 41 and 44 of a predetermined depth, but the reinforcement of the annular projections 53 can solve the problem. The height of the annular projections 53 can be in such a range that the solder 45 can flow into the first seal cavities 42a-42d and the second seal cavities 47a-47d. Also, an upper end of the annular projections 53 can contact an inner wall surface of the outer sleeve 54 in a state of entry of the inner sleeve 51 in the outer sleeve 54. Furthermore, a cutout may be formed through a portion of the annular projections 53 for flow of the solder 45 in an axial direction, so that the first distribution grooves 52a-52c can communicate with one another in the axial direction. It is possible to change the number of the first distribution grooves 52a-52c and their interval.

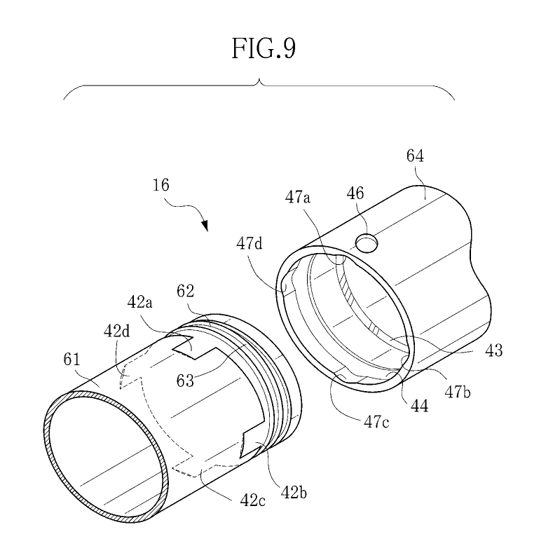

In FIG. 9, a third preferred tube assembly of the invention is illustrated. The first embodiment is repeated but with a difference in that an inner sleeve 61 has a helical distribution groove 62 or distribution channel in contrast with the first distribution groove 41 of the inner sleeve 32.

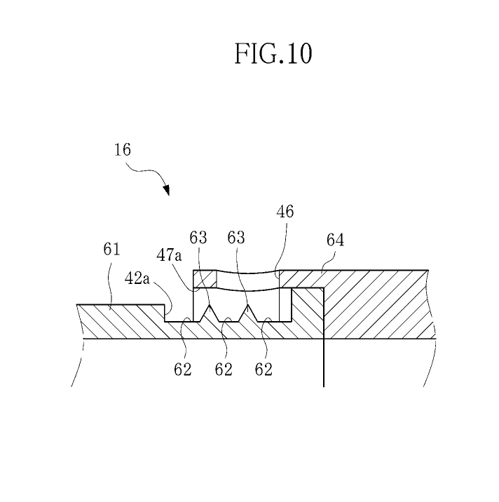

In FIG. 10, a helical projection 63 is defined between turns of the helical distribution groove 62 in a form of a helical thread. An outer sleeve 64 has the flow opening 46. A height of the helical projection 63 is predetermined so as to communicate the flow opening 46 with the first seal cavity 42a. In the same manner as the first embodiment, the inner sleeve 61 is combined with the outer sleeve 64. The solder 45 is injected into the flow opening 46, flows through the helical distribution groove 62 and reaches the first seal cavities 42a-42d.

Thus, strength of soldering can be maintained, because an area of the contact with the solder 45 can be large with the shape of the helical distribution groove 62. Fluidity of the solder 45 can be high because of the inclination of the helical distribution groove 62. Also, the helical projection 63 of the helical distribution groove 62 can function for reinforcement. A problem of a small strength may occur in the first embodiment in that walls are formed with a limited thickness under the first and second distribution grooves 41 and 44 of a predetermined depth, but the reinforcement of the helical projection 63 can solve the problem. The sectional shape of the helical projection 63 may be quadrilateral or trapezoidal in a manner different from the shape according to the embodiment. Also, an upper end of the helical projection 63 may contact an inner wall surface of the outer sleeve 64 in the state of combining the inner sleeve 32 with the outer sleeve 33. Furthermore, a cutout may be formed through a portion of the helical projection 63 for flow of the solder 45 in the axial direction. It is possible to change the number of the turns of the helical distribution groove 62 and their interval.

In the embodiment, the outer sleeve 64 has the second distribution groove 44. However, the outer sleeve 64 may not have the second distribution groove 44.

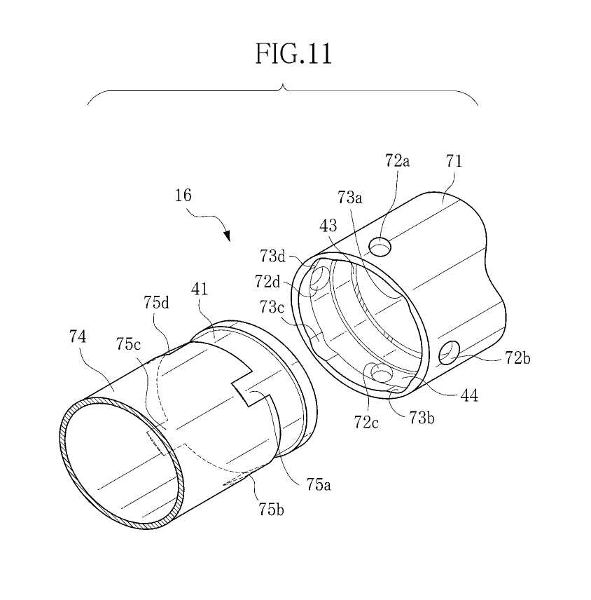

In FIG. 11, a fourth preferred embodiment is illustrated. The first embodiment is repeated but with a difference in that four flow openings 72a, 72b, 72c and 72d or gates are formed in an outer sleeve 71 equiangularly between those. An inner sleeve 74 is combined with the outer sleeve 71. First seal cavities 75a, 75b, 75c and 75d or sub grooves are formed in the outside of the inner sleeve 74. Second seal cavities 73a, 73b, 73c and 73d or sub grooves are formed between the flow openings 72a-72d inside the outer sleeve 71. The first seal cavities 75a-75d are aligned with respectively the second seal cavities 73a-73d when the inner sleeve 74 is mounted in the outer sleeve 71. Elements similar to those of the above embodiments are designated with identical reference numerals.

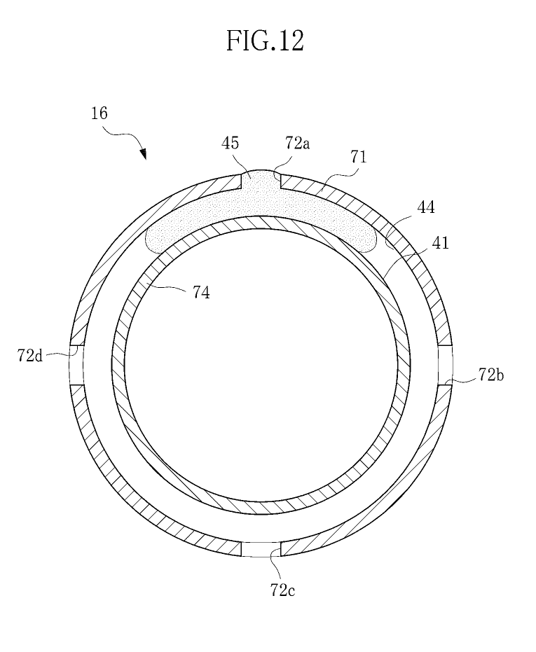

The inner sleeve 74 is mounted in the outer sleeve 71 by aligning the first seal cavities 75a-75d with respectively the second seal cavities 73a-73d. In FIG. 12, the solder 45 is injected in the first and second distribution grooves 41 and 44 through the flow opening 72a. The solder 45 flows circumferentially along the first and second distribution grooves 41 and 44 and then into the first seal cavities 75a and 75d and the second seal cavities 73a and 73d.

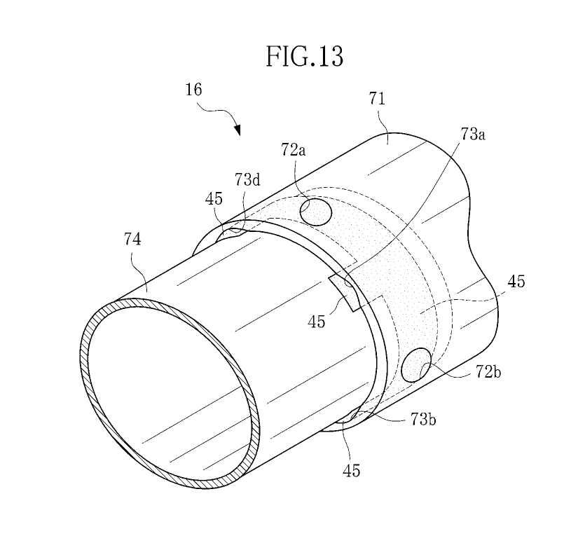

Similarly, the solder 45 is injected into the flow opening 72b and flows into the first seal cavities 75a and 75b and the second seal cavities 73a and 73b. The solder 45 is injected into the flow opening 72c and flows into the first seal cavities 75b and 75c and the second seal cavities 73b and 73c. The solder 45 is injected into the flow opening 72d and flows into the first seal cavities 75c and 75d and the second seal cavities 73c and 73d. In FIG. 13, the first and second distribution grooves 41 and 44 are filled with the solder 45 completely.

In the embodiment, the solder 45 is supplied in four times. Thus, a duration for soldering at each one of the four times is shorter than for soldering of all the solder 45 at one time. It is possible to prevent overheating internal elements with the solder 45. It is possible to check the flow of the solder 45 through the open end areas of the first seal cavities 75a-75d and the second seal cavities 73a-73d. Strength of the soldering can be high.

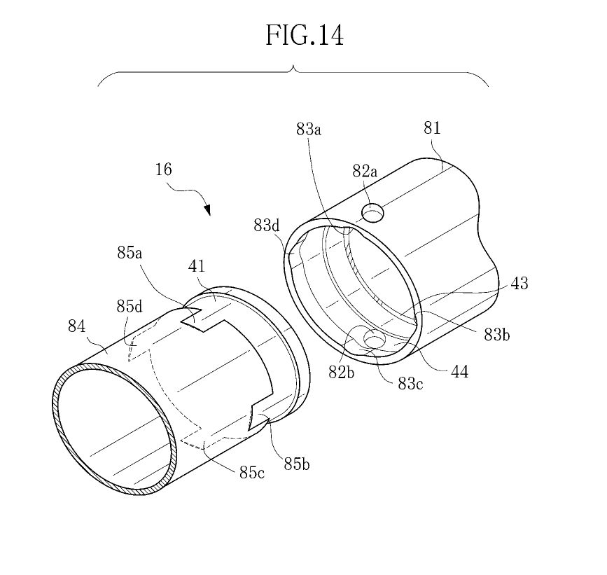

In FIG. 14, a fifth preferred embodiment is illustrated. The first embodiment is repeated but with a difference in that two flow openings 82a and 82b or gates are formed in an outer sleeve 81 symmetrically with reference to the axial direction. An inner sleeve 84 is disposed in the outer sleeve 81. First seal cavities 85a, 85b, 85c and 85d or sub grooves are formed in an outer wall surface of the inner sleeve 84. Second seal cavities 83a, 83b, 83c and 83d or sub grooves are formed in an inner wall surface of the outer sleeve 81, extend from the second distribution groove 44 to a sleeve end of the outer sleeve 81, and are arranged equidistantly. The first seal cavities 85a-85d are aligned with respectively the second seal cavities 83a-83d. Elements similar to those of the above embodiments are designated with identical reference numerals.

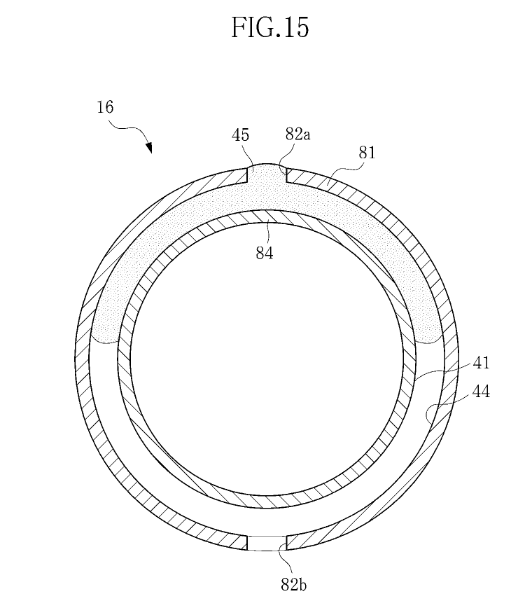

The outer sleeve 81 is mounted on the inner sleeve 84 in the same manner as the above embodiments. In FIG. 15, the solder 45 is injected through the flow opening 82a into the first and second distribution grooves 41 and 44. Then the solder 45 flows to the first seal cavity 85a and the second seal cavity 83a from the first and second distribution grooves 41 and 44. The solder 45 comes to flow circumferentially through the first and second distribution grooves 41 and 44, and enters the first seal cavities 85b and 85d and the second seal cavities 83b and 83d.

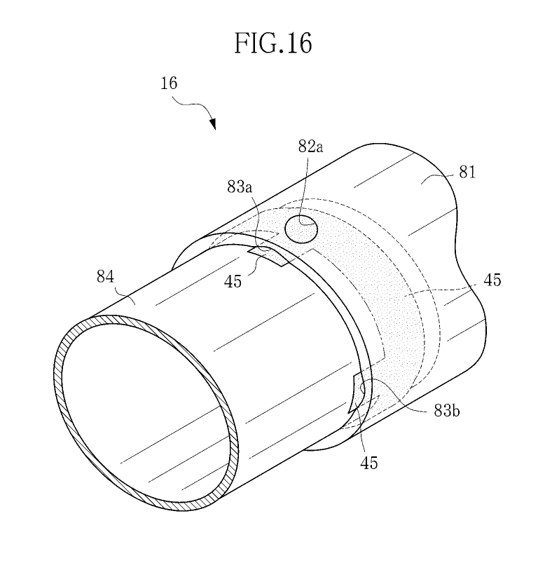

The solder 45 is injected into the flow opening 82b and flows through the first and second distribution grooves 41 and 44 into the first seal cavity 85c and the second seal cavity 83c. Also, the solder 45 flows circumferentially through the first and second distribution grooves 41 and 44, and flows into the first seal cavities 85b and 85d and the second seal cavities 83b and 83d. In FIG. 16, the first and second distribution grooves 41 and 44 are filled with the solder 45 completely.

In the embodiment, the solder 45 is supplied in two times. Thus, a duration for soldering at each one of the two times is shorter than for soldering of all the solder 45 at one time. It is possible to prevent overheating internal elements with the solder 45. It is possible to check the flow of the solder 45 through the open end areas of the first seal cavities 85a-85d and the second seal cavities 83a-83d. Strength of the soldering can be high.

The features of the above embodiments can be combined with one another in a suitable manner for the purpose. The number of the flow openings and seal cavities may be one, or two or more. The positions and arrangement of the flow openings and seal cavities may be changed suitably.

In the above embodiments, the distribution grooves are formed in the circumferential direction in the inner and outer sleeves. However, it is possible to form a plurality of distribution grooves shaped arcuately and arranged circumferentially, for example, two, three or four distribution grooves, according to the number of the flow openings. It is preferable to form seal cavities at ends of respectively the distribution grooves. The seal cavities are kept from clogging with solder overflowing from adjacent flow openings. Soldering can be checked reliably in the seal cavities. Also, a duration for soldering at one time can be shortened. The internal elements can be protected from damages even with heat from the soldering.

In the above embodiments, the steering device is connected to the flexible tube device by the solder joint. However, the feature of the invention can be used for connection between the tip device and the steering device, or between the flexible tube device and the handle, in a structure with the inner and outer sleeves. Also, the feature of the invention can be used for connection of one of the inner and outer sleeves to a proximal link element in the steering device, or to the mesh sleeve or the winding 35 of the strip 34.

In contrast with the solder joint 16 described above, an outer sleeve can be used at the end of the steering device 14, and an inner sleeve can be used at the end of the flexible tube device 15, for soldering according to the invention.

In the above embodiments, the two sleeves are cylindrical. However, sleeves according to the invention may be shaped in a form of an elliptical cylinder, a polygonal prism, a frustum of a cone, and the like.

Also, first and second sleeves with an equal diameter may be used in place of the inner and outer sleeves. A sleeve end of the first sleeve can have a smaller diameter, and can be entered in the second sleeve.

In the embodiments, the sealing material is the flowing solder material 45. However, the sealing material of the invention may be adhesive agent of a liquid state for attaching the inner sleeve to the outer sleeve, or filler, sealant or the like.

Although the present invention has been fully described by way of the preferred embodiments thereof with reference to the accompanying drawings, various changes and modifications will be apparent to those having skill in this field. Therefore, unless otherwise these changes and modifications depart from the scope of the present invention, they should be construed as included therein.

What is claimed is:

1. A tube assembly for an endoscope, including a first section, and a second section disposed to extend from said first section to a proximal side in an axial direction, said tube assembly comprising:
an inner sleeve;
an outer sleeve for receiving one end portion of said inner sleeve in said axial direction, for connection of said second section to said first section;
at least one flow opening formed through said outer sleeve, and opposed to an outer wall surface of said inner sleeve upon receiving said inner sleeve in said outer sleeve;
a distribution groove, formed in at least one of said outer wall surface of said inner sleeve and an inner wall surface of said outer sleeve, to extend from said flow opening according to a circumferential direction;
at least one seal cavity formed in at least one of said outer wall surface of said inner sleeve and said inner wall surface of said outer sleeve, to extend from said distribution groove to a sleeve end of said outer sleeve in an externally open form;
sealing material, supplied in said flow opening, charged in said distribution groove and said seal cavity, for attaching said outer sleeve to said inner sleeve.

2. A tube assembly as defined in claim 1, wherein said distribution groove is annular.

3. A tube assembly as defined in claim 1, wherein said distribution groove includes:
a first distribution groove formed in said outer wall surface of said inner sleeve;
a second distribution groove, formed in said inner wall surface of said outer sleeve, and disposed in alignment with said first distribution groove upon mounting said inner sleeve in said outer sleeve.

4. A tube assembly as defined in claim 3, wherein said seal cavity includes:
a first seal cavity formed in said inner sleeve to extend from said first distribution groove in said axial direction;
a second seal cavity formed in said outer sleeve to extend along said first seal cavity from said second distribution groove to a sleeve end of said outer sleeve.

5. A tube assembly as defined in claim 4, wherein said first seal cavity includes an open end area disposed outside said sleeve end of said outer sleeve.

6. A tube assembly as defined in claim 1, wherein said distribution groove includes plural distribution grooves arranged adjacently to one another in said axial direction.

7. A tube assembly as defined in claim 1, wherein said distribution groove is in a helical shape with plural turns.

8. A tube assembly as defined in claim 1, wherein said at least one flow opening is a plurality of flow openings formed in said outer sleeve and arranged substantially equidistantly in said circumferential direction thereof.

9. A tube assembly as defined in claim 1, wherein said at least one seal cavity is plural seal cavities of which a number is equal to or larger than a number of said flow opening.

10. A tube assembly as defined in claim 9, wherein said at least one flow opening is plural flow openings, and said seal cavities are offset from said flow openings in said circumferential direction of said outer sleeve.

11. A tube assembly as defined in claim 9, wherein said seal cavities are aligned with said flow opening in said axial direction.

12. A tube assembly as defined in claim 1, wherein said first section is a steering device, and said second section is a flexible tube device.

13. A tube assembly as defined in claim 12, wherein said inner sleeve is disposed at an end of said steering device, and said outer sleeve is disposed at an end of said flexible tube device.

14. A tube assembly as defined in claim 13, wherein said steering device includes first to Nth link elements, arranged serially in said axial direction toward said flexible tube device, and connected with one another movably;
said inner sleeve is said Nth link element.

15. A tube assembly as defined in claim 13, further comprising a cover sleeve, mounted on at least said steering device, for covering said inner and outer sleeves.

16. An attaching method for an endoscope having a tube assembly, said tube assembly including a tip device having an internal element, a steering device, mounted on a proximal side of said tip device, for steering operation, and a flexible tube device disposed to extend from said steering device to a proximal side in an axial direction, wherein an inner sleeve is used to constitute a proximal end of said steering device, an outer sleeve is used to constitute a distal end of said flexible tube device, one end portion of said inner sleeve is receivable in said outer sleeve in said axial direction, for connection of said flexible tube device to said steering device, said attaching method comprising steps of:
forming at least one flow opening through said outer sleeve, said flow opening being opposed to an outer wall surface of said inner sleeve upon receiving said inner sleeve in said outer sleeve;
forming a distribution groove in at least one of said outer wall surface of said inner sleeve and an inner wall surface of said outer sleeve, to extend from said flow opening according to a circumferential direction;
forming at least one seal cavity to extend from said distribution groove to a sleeve end of said outer sleeve in an externally open form upon receiving said inner sleeve in said outer sleeve;
penetrating a cable and an elongated element through said steering device and said flexible tube device in connection with said internal element;
mounting said inner sleeve in said outer sleeve upon penetrating said cable and said elongated element through said flexible tube device;
after said mounting step, supplying sealing material in said flow opening, to charge said distribution groove and said seal cavity with said sealing material.

17. An attaching method as defined in claim 16, wherein said sealing material is flowing solder material.
